(19) United States
(12) Patent Application Publication
(10) Pub. No.: US 2015/0112221 A1
(43) Pub. Date: Apr. 23, 2015

(54) METHOD AND DEVICE FOR MEASURING THE CONCENTRATION OF NITROGEN MONOXIDE IN THE RESPIRATORY AIR OF A PATIENT

(71) Applicant: Robert Bosch GmbH, Stuttgart (DE)

(72) Inventors: Oliver von Sicard, Muenchen (DE); Erhard Magori, Feldkirchen (DE); Roland Pohle, Herdweg (DE); Maximilian Fleischer, Hoehenkirchen (DE)

(21) Appl. No.: 14/512,399

(22) Filed: Oct. 11, 2014

(30) Foreign Application Priority Data
Oct. 17, 2013 (DE) 10 2013 221 061.2

(51) Int. Cl. A61B 5/08; A61B 5/097

(52) U.S. Cl. CPC A61B 5/082; A61B 5/097
USPC 600/532

(57) ABSTRACT

A device for measuring the concentration of nitrogen monoxide in the respiratory air of a patient includes a nitrogen dioxide sensor and a converter. The sensor is arranged between an inlet opening and an outlet opening of the device. The converter, for oxidation of nitrogen monoxide to nitrogen dioxide, is arranged between the inlet opening and the sensor such that the device can be switched to at least two states. In a first state, a fluidic connection is present between the inlet opening and the sensor but does not lead through the converter. In a second state, a fluidic connection is present between the inlet opening and the sensor and leads through the converter.

This application claims priority under 35 U.S.C. §119 to patent application number DE 10 2013 221 061.2, filed on Oct. 17, 2013 in Germany, the disclosure of which is incorporated herein by reference in its entirety.

### BACKGROUND

The present disclosure relates to a device for measuring the concentration of nitrogen monoxide in the respiratory air of a patient. The present disclosure further relates to a method for measuring the concentration of nitrogen monoxide in the respiratory air of a patient, in particular using the device according to the disclosure.

A non-invasive way of detecting diseases and metabolic disorders is to measure marker gases in the exhaled air of humans. Such measurements can be used both in screening and also in differential diagnosis and for optimization of therapy. For example, a measurement of nitrogen monoxide in exhaled air can be used for monitoring of asthma therapy, for differential diagnosis of COPD (chronic obstructive pulmonary disease), and for detection of lung tumors, tuberculosis and pneumonia.

Measuring the concentration of nitrogen monoxide in respiratory gas is an important means of optimizing the treatment of asthma. Until recently, inexpensive nitrogen monoxide sensors with the required sensitivity in the ppb range were unavailable on the market. A newly developed nitrogen dioxide sensor based on suspended gate field-effect transistor technology (SGFET), and containing a porphin dye as gas-sensitive layer, meets these requirements. However, a conversion module for converting the nitrogen monoxide (NO) in the respiratory gas to nitrogen dioxide (NO₂), which can be detected by the sensor, has to be placed upstream of the sensor. Moreover, before each measurement of the nitrogen dioxide concentration, the sensor briefly requires a reference gas, free of nitrogen dioxide, in order to generate a sensor baseline. Changes in the nitrogen dioxide concentration are then detected. EP 1 384 069 B1 describes such a device for quantitative measurement of nitrogen oxides in exhaled air. In order to flush the sensor with ambient air before each measurement, a pump is needed that pumps ambient air into the measuring chamber of the device. Since ambient air can contain a nitrogen dioxide concentration of up to several 10 ppb, depending on the environmental conditions, this air first of all has to be pumped through an active carbon filter. In doing this, the filter is used up. In terms of its moisture content and its temperature, however, the reference air thus prepared differs greatly from air exhaled by humans, which has a temperature of ca. 35° C. and a relative humidity of 100%. For this reason, the sensor signal is not influenced just by a change in nitrogen dioxide concentration.

### SUMMARY

The device according to the disclosure for measuring the concentration of nitrogen monoxide in the respiratory air of a patient comprises a nitrogen dioxide sensor, which is arranged between an inlet opening and an outlet opening of the device, and a converter for oxidation of nitrogen monoxide to nitrogen dioxide, which converter is arranged between the inlet opening and the nitrogen dioxide sensor such that the device can be switched to at least two states, wherein, in a first state, a fluidic connection is present between the inlet opening and the nitrogen dioxide sensor but does not lead through the converter, and, in a second state, a fluidic connection is present between the inlet opening and the nitrogen dioxide sensor and leads through the converter. In the first state, preferably no fluidic connection between the inlet opening and the nitrogen dioxide sensor is present that does not lead through the converter. In the second state, preferably no fluidic connection between the inlet opening and the nitrogen dioxide sensor is present that leads through the converter. This device allows the nitrogen monoxide concentration in the respiratory air of a patient to be measured by means of the method according to the disclosure. In this method, a first respiratory air sample of the patient is brought into contact with a nitrogen dioxide sensor, a first signal of the nitrogen dioxide sensor is detected, the nitrogen dioxide sensor is referenced by means of the first signal, nitrogen monoxide in a second respiratory air sample of the patient is oxidized to nitrogen dioxide, the second respiratory air sample is brought into contact with the nitrogen dioxide sensor, a second signal of the nitrogen dioxide sensor is detected, and the concentration of nitrogen monoxide in the respiratory air of the patient is determined from the second signal. According to the disclosure, a respiratory air sample is understood as a quantity of the patient's respiratory air, or a part of a quantity of the patient's respiratory air, that is being examined in the method according to the disclosure. It is possible for the patient to provide the first respiratory air sample and the second respiratory air sample without interrupting his exhalation. Depending on environmental conditions, the air exhaled by humans contains no nitrogen dioxide. Therefore, the device according to the disclosure does not require an active carbon filter of limited service life, nor does it require a pump for flushing the nitrogen dioxide sensor. Moreover, in the method according to the disclosure, the nitrogen dioxide-free reference gas that is made available for the nitrogen dioxide sensor is exhaled human air, i.e. a gas having the same residual gases and the same properties, in particular temperature and humidity, as the respiratory air sample to be examined during the nitrogen dioxide measurement for determining the concentration of nitrogen monoxide in the respiratory air.

In a preferred embodiment of the device according to the disclosure, an exchange element is arranged between the inlet opening and the nitrogen dioxide sensor and comprises the converter and a connecting element which cannot perform oxidation of nitrogen monoxide to nitrogen dioxide, wherein the converter or the connecting element can be moved alternately into the fluidic connection between the inlet opening and the nitrogen dioxide sensor. In a preferred embodiment of the method according to the disclosure, this means that, after the first signal is detected, the converter for oxidation of nitrogen monoxide to nitrogen dioxide can be moved into the fluidic connection between the inlet opening, for admission of respiratory air of the patient, and the nitrogen dioxide sensor, and that the converter can be moved out of the fluidic connection after the second signal is detected. For this purpose, use is particularly preferably made of the exchange element arranged in the fluidic connection, which element comprises the converter and the connecting element, which can perform no oxidation of nitrogen monoxide to nitrogen dioxide, such that, when the converter is moved out of the fluidic connection, the connecting element is moved into the fluidic connection.

In another preferred embodiment of the device according to the disclosure, two fluidic connections are arranged between the inlet opening and the nitrogen dioxide sensor and can each be closed by a valve, in particular independently of each other, wherein the converter is arranged in one of these connections, and, in the other connection, there is no element configured to perform oxidation of nitrogen monoxide to nitrogen dioxide. In a preferred embodiment of the method according to the disclosure, this means that, after the first signal is detected, a first fluidic connection between an inlet opening, for admission of respiratory air of the patient, and the nitrogen dioxide sensor can be closed, and a second fluidic connection, in which a converter for oxidation of nitrogen monoxide to nitrogen dioxide is arranged, can be opened between the inlet opening and the nitrogen dioxide sensor, and that, after the second signal is detected, the second fluidic connection can be closed and the first fluidic connection opened. It is particularly preferable that the opening and closing of the first fluidic connection and of the second fluidic connection take place by means of valves. In a very particularly preferred embodiment of the method, a valve is arranged upstream of the converter. In another very particularly preferred embodiment of the method, a valve is arranged downstream of the converter. It can in particular be a three-way valve, which permits the opening and closing of the first fluidic connection and also the opening and closing of the second fluidic connection.

In another preferred embodiment, the device according to the disclosure comprises a flow divider which is configured such that a first part of a stream of fluid between the inlet opening and the outlet opening is routed through the converter, and a second part of the stream of fluid is not routed through the converter, wherein the ratio between the first part and the second part can be changed by a user. In a preferred embodiment of the method according to the disclosure, this means that, before the first signal of the nitrogen dioxide sensor is detected, it is possible to generate a large flow of respiratory air through the converter and a small flow of respiratory air past the converter and, before the second signal of the nitrogen dioxide sensor is detected, it is possible to generate a small flow of respiratory air through the converter and large flow of respiratory air past the converter.

It is preferable that the nitrogen dioxide sensor is arranged in a measuring chamber, and that a valve is arranged in a fluidic connection between the measuring chamber and the outlet opening. In a preferred embodiment of the method according to the disclosure, this means that, during the detection of the first signal and during the detection of the second signal, a fluidic connection between the measuring chamber and an inlet opening, for admission of respiratory air of the patient into the measuring chamber, can be closed, and a fluidic connection between the measuring chamber and an outlet opening for discharging respiratory air of the patient from the measuring chamber can be closed. In this way, the measurement of the first signal and the measurement of the second signal cannot be adulterated by air flowing onto the nitrogen dioxide sensor from outside.

### BRIEF DESCRIPTION OF THE DRAWINGS

Illustrative embodiments of the disclosure are shown schematically in the drawings and are explained in more detail in the description below.

FIG. 1 is a schematic view of a device according to one embodiment of the disclosure.

FIG. 2 is a schematic view of a device according to another embodiment of the disclosure.

FIG. 3 shows the time profile of a first nitrogen dioxide sensor signal in a method according to one embodiment of the disclosure.

FIG. 4 shows the time profile of a second nitrogen dioxide sensor signal in a method according to one embodiment of the disclosure.

### DETAILED DESCRIPTION

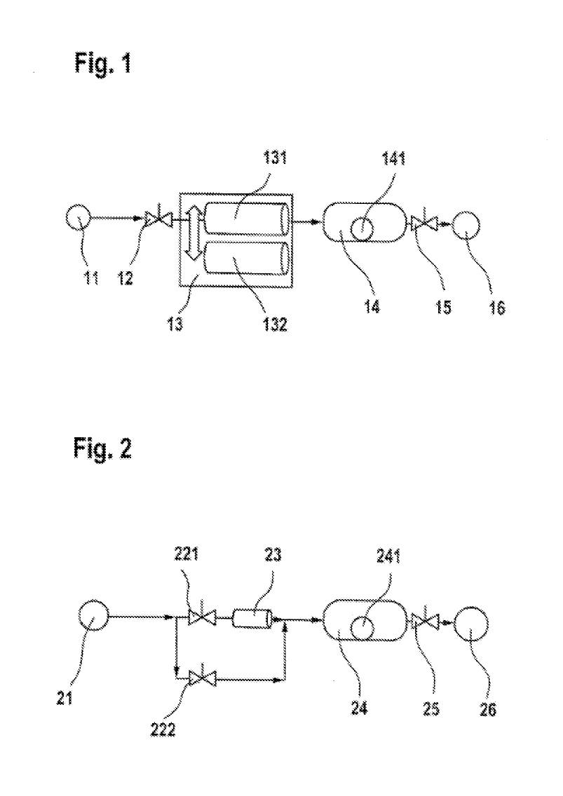

A first embodiment of a device according to the disclosure for measuring the concentration of nitrogen monoxide in the respiratory air of a patient is shown schematically in FIG. 1. An inlet opening 11 is fluidically connected to an inlet valve 12. The inlet valve 12 is fluidically connected to a measuring chamber 14. An exchange element 13 is arranged in the fluidic connection between the inlet valve 12 and the measuring chamber 14. This exchange element 13 contains a converter 131 and a connecting element 132. The converter contains potassium permanganate on zeolite as carrier material. The connecting element 132 corresponds in size to the converter 131 but is empty. The exchange element 13 is configured such that either the converter 131 or the connecting element 132 can be moved into the fluidic connection between the inlet valve 12 and the measuring chamber 14. The measuring chamber 14 contains an SGFET nitrogen dioxide sensor 141. The measuring chamber 14 is fluidically connected to an outlet opening 16, this fluidic connection leading through an outlet valve 15.

In a first embodiment of the method according to the disclosure, a patient firstly blows a respiratory gas portion of 100 to 500 ml through the inlet opening 11, the inlet valve 12 and the connecting element 132 into the measuring chamber 14. The inlet valve 12 and the outlet valve 15 are then closed and the nitrogen dioxide sensor 141 detects a first signal. Thereafter, the two valves 12, 15 are opened again, and the converter 131 and the connecting element 132 swap positions in the exchange element 13. The patient now blows a second respiratory gas portion through the inlet opening 11, the inlet valve 12 and the converter 131 into the measuring chamber 14. This second respiratory gas portion is brought into contact with the potassium permanganate in the converter 131. In this way, nitrogen monoxide contained in the respiratory gas is oxidized to nitrogen dioxide:

3 NO+2 KMnO₄+H₂O- >3 NO₂+2 MnO₂+2 KOH

The first respiratory gas portion is forced out of the measuring chamber 14 by the second respiratory gas portion and leaves the device by way of the outlet valve 15 and the outlet opening 16. The valves 12, 15 are then closed, and the sensor 141 detects a second nitrogen dioxide signal. Thereafter, the valves 12, 15 are opened again. From the difference between the first signal and the second signal of the nitrogen dioxide sensor 141, it is possible to determine the contribution made to the measured nitrogen dioxide by the nitrogen dioxide that is obtained by oxidation of nitrogen monoxide to nitrogen dioxide. The quantity of this nitrogen dioxide corresponds to the quantity of nitrogen monoxide contained in the second respiratory gas portion.

A device according to a second embodiment of the disclosure is shown schematically in FIG. 2. An inlet opening 21 is fluidically connected to a measuring chamber 24. A first inlet valve 221 and a KMnO₄/zeolite converter 23 are arranged in this connection. Between the inlet opening 21 and the first inlet valve 221, a path of the fluidic connection branches off which is routed through a second inlet valve 222 and ends between the converter 23 and the measuring chamber 24. The measuring chamber 24 contains an SGFET nitrogen dioxide sensor 241. The measuring chamber 24 is fluidically connected to an outlet opening 26, with an outlet valve 25 being arranged in this fluidic connection.

The first inlet valve 221 is initially closed and the second inlet valve 222 and the outlet valve 25 are opened. In a second embodiment of the method according to the disclosure, a patient can now blow a first respiratory gas portion through the inlet opening 21 and the second inlet valve 222 into the measuring chamber 24. The second inlet valve 222 and the outlet valve 25 are now closed, and the nitrogen dioxide sensor 241 detects a first signal. The outlet valve 25 and the first inlet valve 221 are then opened. Thereafter, the patient blows a second respiratory gas portion through the inlet opening 21, the first inlet valve 221 and the converter 23 into the measuring chamber 24 and in so doing forces the first respiratory gas portion out of the measuring chamber 24 by way of the outlet valve 25 and the outlet opening 26. Thereafter, the two opened valves 221, 25 are closed, and the nitrogen dioxide sensor 241 detects a second signal. Finally, the second inlet valve 222 and the outlet valve 25 are opened again. The nitrogen monoxide content in the second respiratory gas portion of the patient is determined from the first and second sensor signals in the same way as in the first embodiment of the method according to the disclosure.

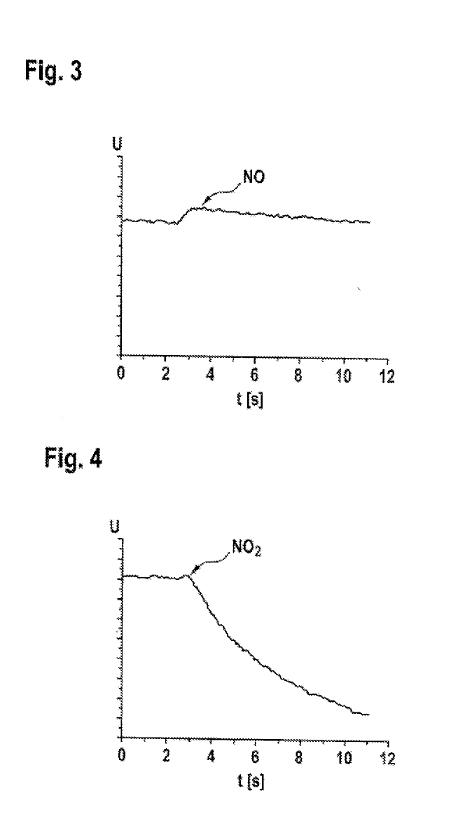

FIG. 3 shows the profile of a sensor voltage signal U over time t, after a first respiratory gas portion has been blown, at a point NO, into a measuring chamber 14, 24 of the first or second embodiment of the device according to the disclosure. This respiratory gas portion contains 30 ppb of nitrogen monoxide, which does not however lead to any relevant change of the voltage signal U. FIG. 4 shows the time profile of the sensor voltage signal U after the second respiratory gas portion has been blown into the measuring chamber 14, 24 of a device according to the first and the second embodiments of the disclosure. After 30 ppb of nitrogen dioxide, generated by oxidation of 30 ppb of nitrogen monoxide in the converter 131, 23, has come into contact at point NO₂ with the nitrogen dioxide sensor 141, 241, a clear voltage can be observed. By subtraction of the two nitrogen dioxide sensor signals from FIGS. 3 and 4, it is possible to determine the nitrogen dioxide quantity of the second respiratory gas portion that corresponds to its nitrogen monoxide quantity.

What is claimed is:

1. A device for measuring the concentration of nitrogen monoxide in the respiratory air of a patient, comprising:
a nitrogen dioxide sensor arranged between an inlet opening and an outlet opening of the device; and
a converter configured to oxidize nitrogen monoxide to nitrogen dioxide, the converter arranged between the inlet opening and the nitrogen dioxide sensor such that the device is configured to be switched to at least two states,
wherein, in a first state, a fluidic connection between the inlet opening and the nitrogen dioxide sensor does not lead through the converter, and
wherein, in a second state, the fluidic connection between the inlet opening and the nitrogen dioxide sensor leads through the converter.

2. The device according to claim 1, wherein:
in the first state, no fluidic connection between the inlet opening and the nitrogen dioxide sensor is present that leads through the converter, and
in the second state, no fluidic connection between the inlet opening and the nitrogen dioxide sensor is present that does not lead through the converter.

3. The device according to claim 1, further comprising:
an exchange element arranged between the inlet opening and the nitrogen dioxide sensor, the exchange element including the converter and a connecting element, the connecting element not being configured to perform oxidation of nitrogen monoxide to nitrogen dioxide,
wherein at least one of the converter and the connecting element is configured to be moved alternately into the fluidic connection between the inlet opening and the nitrogen dioxide sensor.

4. The device according to claim 1, further comprising:
two fluidic connections arranged between the inlet opening and the nitrogen dioxide sensor,
wherein each of the two fluidic connections is configured to be closed by a valve,
wherein the converter is arranged in a first of the two fluidic connections, and
wherein, in a second of the two fluidic connections, no element configured to perform oxidation of nitrogen monoxide to nitrogen dioxide is arranged.

5. The device according to claim 1, further comprising:
a flow divider configured such that a first part of a stream of fluid between the inlet opening and the outlet opening is routed through the converter and a second part of the stream of fluid is not routed through the converter,
wherein the ratio between the first part and the second part is changeable by a user.

6. The device according to claim 1, wherein:
the nitrogen dioxide sensor is arranged in a measuring chamber, and
a valve is arranged in a fluidic connection between the measuring chamber and the outlet opening.

7. A method for measuring the concentration of nitrogen monoxide in the respiratory air of a patient, the method comprising:
bringing a first respiratory air sample of the patient into contact with a nitrogen dioxide sensor;
detecting a first signal of the nitrogen dioxide sensor;
referencing the nitrogen dioxide sensor by the first signal;
oxidizing nitrogen monoxide in a second respiratory air sample of the patient to nitrogen dioxide;
bringing the second respiratory air sample into contact with the nitrogen dioxide sensor;
detecting a second signal of the nitrogen dioxide sensor; and
determining the concentration of nitrogen monoxide in the respiratory air of the patient from the second signal.

8. The method according to claim 7, further comprising:
after detecting the first signal, moving a converter configured to oxidize nitrogen monoxide to nitrogen dioxide into a fluidic connection between an inlet opening, for admission of respiratory air of the patient, and the nitrogen dioxide sensor; and
after detecting the second signal, moving the converter out of the fluidic connection.

9. The method according to claim 8, wherein:
an exchange element is arranged in the fluidic connection, the exchange element including the converter and a connecting element, the connecting element not being configured to perform oxidation of nitrogen monoxide to nitrogen dioxide, and
the connecting element is moved into the fluidic connection when the converter is moved out of the fluidic connection.

10. The method according to claim 7, further comprising:
after detecting the first signal, closing a first fluidic connection and opening a second fluidic connection; and
after detecting the second signal, closing the second fluidic connection and opening the first fluidic connection,
wherein the first fluidic connection is between an inlet opening, for admission of respiratory air of the patient, and the nitrogen dioxide sensor, and
wherein the second fluidic connection, in which a converter configured to oxidize nitrogen monoxide to nitrogen dioxide is arranged, is between the inlet opening and the nitrogen dioxide sensor.

11. The method according to claim 10, wherein the first fluidic connection and the second fluidic connection are opened and closed by valves.

12. The method according to claim 7, wherein:
the nitrogen dioxide sensor is arranged in a measuring chamber, and
during the detection of the first signal and during the detection of the second signal, a fluidic connection between the measuring chamber and an inlet opening, for admission of respiratory air of the patient into the measuring chamber, is closed, and a fluidic connection between the measuring chamber and an outlet opening for discharging respiratory air of the patient from the measuring chamber is closed.
